(19) United States
(12) Patent Application Publication
(10) Pub. No.: US 2023/0293048 A1
(43) Pub. Date: Sep. 21, 2023

(54) Virtual Immersive Sensorimotor Training System for Improving Functional Performance

(71) Applicant: University of Mississippi Medical Center, Jackson, MS (US)

(72) Inventor: Jennifer C. Reneker, Clinton, MS (US)

(21) Appl. No.: 18/141,324

(22) Filed: Apr. 28, 2023

Related U.S. Application Data
(63) Continuation of application No. 16/786,983, filed on Feb. 10, 2020, now abandoned.
(60) Provisional application No. 62/803,050, filed on Feb. 8, 2019.

(51) Int. Cl. A61B 5/11; G06F 3/01; G02B 27/01

(52) U.S. Cl. CPC A61B 5/1124; A61B 5/1126; G02B 27/017; G06F 3/013; A61B 2505/09

(57) ABSTRACT

Computer systems and methods of using computer systems to provide a virtual immersive environment for training sensorimotor control of a user and for improving functional performance of a user; the systems and methods being used to treat neurological impairment associated with a concussion or other injury, neurological impairment associated with aging, for training to improve functional performance above baseline, or for increasing sensorimotor control to prevent injury.

### CROSS-REFERENCE TO RELATED APPLICATIONS

This application claims priority to, and the benefit of, U.S. Provisional Pat. Application Serial No. 62/803,050, filed on Feb. 8, 2019 and titled “An Immersive Visual-Vestibular Interface Sensorimotor-System and Methods to Improve Sensorimotor Control,” which is incorporated herein by this reference in its entirety.

### BACKGROUND

The skilled movements of those (such as athletes, military service members, and emergency first responders) participating in fast paced and competitive environments require integration of high levels of coordination, fast reaction times, and good balance and agility, all while navigating the trajectory of other humans and/or objects (such as a ball). These abilities are made possible through subconscious integration of disparate sensory information, with contributions from multiple sub-systems, in a manner that permits appropriate motor output from the central nervous system. This process of integration is known collectively as sensorimotor control.

Sensory information includes information from the vestibular, visual, and mechanoreceptor systems. The vestibular system senses angular and linear acceleration, providing feedback and eliciting ocular and postural reflexes. The visual system is used to plan movements in a feed-forward manner. The mechanoreceptor system provides information across the joints and muscles in the body to permit kinesthetic awareness. This sensory information directs the efferent output necessary to complete the intended action through precise motor control. Just as honing of other skills such as neuromuscular strength, explosive power, speed, and endurance can lead to greater functional performance, higher acuity of the sensorimotor control system can improve overall functional performance of a subject.

Physical activities such as those related to sports, military, policing, and the like can lead to physical injuries, which limit participation and decrease performance. In particular, concussion has emerged as one of the most concerning sports injuries, with upwards of 3.8 million traumatic brain injuries (TBI), predominantly mild TBl/concussion, occurring annually in the United States. In the US military, annual concussion incidence is variable, depending on engagement in combat, branch of the military, and specific duties, but nonetheless represents a significant issue. Mechanisms of injury in military environments for concussion comprise varying etiologies, including those from blunt forces and high-explosive blast forces.

According to data from the National Collegiate Athletic Association Injury Surveillance Program (NCAA-ISP), in the US, aside from concussion, ankle sprains and anterior-cruciate ligament injuries are the most common orthopedic conditions occurring each year as a result of sports participation. Musculoskeletal injuries also commonly befall US service members, with nearly 1.6 million such injuries occurring each year.

When a concussion or musculoskeletal injury occurs, a primary goal is to return to play or service activity. Generally speaking, concussion recovery is established once there is dissipation of self-reported symptoms (including headaches, dizziness, noise, and light sensitivity, emotional concerns, sleep disturbances, etc.) at rest and with activity. However, lingering detrimental effects may remain even after a patient has been determined, under conventional metrics, to have recovered from the concussion injury.

For other injuries, the absence of pain and return of normalized movement patterns needed for function are key indicators of recovery. In order for musculoskeletal injuries to be appropriately yet safely challenged, physical therapy intervention is routinely utilized to provide treatment as well as guidance related to return to activity.

At this time, however, physical therapy is not routinely utilized after a concussion to establish fitness for return to play, work, or duty. This is due at least in part to limitations of conventional physical therapy. For example, the training and/or diagnostic activities conventionally utilized to measure sensorimotor control are difficult to objectively quantify, making tracking of patient progress over time and/or across different caregivers difficult.

Further, inherent physical constraints severely limit the type and extent of the training activities that the patient can undergo. For example, it is essentially impossible to control 100% of the patient’s visual field, even in a controlled environment. Real-world objects used in such therapy exercises, such as visual target objects, can only be moved and utilized in a limited number of ways and are of course subject to normal real-world physical constraints. Conventional application of such exercises also requires the caregiver to be present to apply the training/exercise to the patient and make qualitative assessments.

There is thus an ongoing need for better treatment of concussion injuries in a manner that provides better recovery to pre-injury levels of sensorimotor control and functional performance or better, and for treatments that better prevent concussion injuries.

### SUMMARY

Disclosed are computer systems and methods of using computer systems to provide a virtual immersive environment for training sensorimotor control of a user and for improving functional performance of a user. The systems and methods may be utilized to treat neurological impairment associated with a concussion or other injury, neurological impairment associated with aging, for training to improve functional performance above baseline, or for increasing sensorimotor control to prevent injury.

In one embodiment, a computer system includes a head-mounted display for immersing a user in a virtual immersive environment, and one or more position/motion sensors for recording movement data of a user within the virtual immersive environment. The computer system operates to display a virtual immersive environment to the user by way of the head-mounted display, and to provide instructions directing the user to execute one or more sensorimotor activities within the virtual immersive environment. The computer system records movement data of the user associated with performance of the one or more sensorimotor activities. Based on the recorded movement data, the computer system determines a baseline sensorimotor activity metric of the user indicating the user’s baseline proficiency in executing the one or more sensorimotor activities.

The computer system then provides additional instructions directing the user to repeat execution of the one or more sensorimotor activities within the virtual immersive environment, and records further movement data of the user during the repeated execution of the one or more sensorimotor activities. The computer system may be controlled, for example, by a physical therapist, other healthcare provider, trainer, or the like. Based on the recorded further movement data, the computer system determines a trained sensorimotor activity metric of the user indicating the user’s proficiency in executing the one or more sensorimotor activities following the additional execution of the one or more sensorimotor activities, and compares the trained sensorimotor activity metric to the baseline sensorimotor activity metric to indicate the user’s improvement (or lack thereof) in performing the one or more sensorimotor activities. In some embodiments, the computer system and/or method therefore utilizes sensorimotor training activities within a virtual immersive environment to beneficially increase the sensorimotor control of the user.

In some embodiments, the computer system and/or method beneficially improve functional performance of a user in addition to increasing the sensorimotor control of the user. In one embodiment, for example, upon determining an increase in proficiency in performing at least one sensorimotor activity within the virtual immersive environment, the computer system may record a trained functional performance metric of a user, and may compare the trained functional performance metric to a baseline functional performance metric (recorded prior to training with the one or more sensorimotor activities) to illustrate improved functional performance of the user.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an exemplary computing environment in which a virtual immersive environment may be generated for providing sensorimotor control training to a user;

FIG. 2 illustrates an exemplary method of using the computer systems of FIG. 1 to generate a virtual immersive environment in which the user may perform one or more sensorimotor activities, obtaining movement data indicating position and movement of the user in response to sensory stimuli within the virtual environment, and providing sensorimotor metrics indicative of the user’s ability to execute the one or more sensorimotor activities; and

FIGS. 3A-3F illustrate various exemplary virtual immersive environments that may be presented to the user for various corresponding sensorimotor or functional performance activities.

### DETAILED DESCRIPTION

### Introduction: Concussions and Injury Risk

A key concern with return to activity is the risk of re-injury. In sports, for example, the inventors discovered that athletes who have suffered one concussion are at a higher risk for one or more subsequent concussions. In total, 17 meta-analyses were completed. Based on the resulting I², nine utilized random-effects models and eight utilized fixed effects models.

In service members and athletes, seventeen studies reported an Odds Ratio including a total of nearly 4.7 million participants. Results are presented in Table 1. The pooled Odds Ratio of those with a previous concussion sustaining a second injury is 2.55 compared to those with no previous concussion. Four studies reported a hazard ratio including a total of 538,822 participants. The pooled Hazard Ratio for those with a previous concussion sustaining a secondary injury is 1.40 compared to those with no previous concussion.

TABLE 1

Meta-Analyses for Risk of Any Injury
Odds Ratio

| Outcome | Studies (n) | Participants (n) | I-Square | Estimate (95% Cl) |
|---|---|---|---|---|
| Any injury | 17 | Athletes and Military Concussion = 35,559 No concussion = ~4,700,000 | 95.95 | 2.55 (1.85, 3.52) |
| Any injury | 14 | Athletes Concussion = 22,876 No concussion = ~4,700,000 | 94.38 | 2.75 (1.90, 3.98) |
| Any injury | 3 | Military Concussion = 12,683 No concussion = 13,134 | 74.81 | 1.54 (1.07, 2.21) |
| Hazard Ratio | | | | |
| Any injury | 4 | Athletes and Military Concussion = 16,763 No concussion = 522,059 | 2.62 | 1.40 (1.32, 1.48) |
| Any injury | 2 | Athletes Concussion = 176 No concussion = 1,709 | 0.00 | 1.69 (1.29, 2.20) |
| Any injury | 2 | Military Concussion = 16,587 No concussion = 520,350 | 7.15 | 1.39 (1.31, 1.47) |
| Any injury | 7 | Athletes Concussion = 1,047 No concussion = 26,224 | 0.00 | 1.72 (1.49, 1.98) |

In athletes only, fourteen studies reported an Odds Ratio including a total of nearly 4.7 million participants. Considering the odds of all injury in athletes, the pooled Odds Ratio of those with a previous concussion sustaining a second injury is 2.75 compared to athletes with no history of concussion. Two studies reported a Hazard Ratio including a total of 1,885 participants. The pooled Hazard Ratio for a second injury is 1.69 in athletes with a history of concussion compared to athletes with no history of concussion. Seven studies reported a rate ratio including a total of 27,271 athletes. The pooled Rate Ratio for athletes with a previous concussion sustaining a secondary injury is 1.72 compared to those with no previous concussion.

In service members only, three studies reported an odds ratio including a total of 25,817 participants. The pooled Odds Ratio for service members with a previous concussion sustaining a secondary injury is 1.54 compared to those with no previous concussion. Two studies reported a Hazard Ratio with a total of 536,937 service members. The summary Hazard Ratio for any injury is 1.39 for those with a previous concussion compared to no previous concussion.

For the meta-analysis describing risk of concussion in service members and athletes with and without a history of a concussion, seven studies reported an Odds Ratio including a total of 36,400 participants. In Table 2, the summary estimate of those with a previous concussion sustaining a secondary concussion is 3.73 compared to those with no history of concussion.

TABLE 2

Meta-Analyses for Risk of Concussion
Odds Ratio

| Outcome | Studies (n) | Participants (n) | I-Square | Estimate (95% Cl) |
|---|---|---|---|---|
| Concussion | 7 | Athletes and Military Concussion = 3,087 No concussion = 33,313 | 85.80 | 3.73 (2.41, 5.78) |
| Concussion | 3 | Athletes Concussion = 1,926 No concussion = 31,701 | 78.76 | 4.44 (2.90, 6.79) |
| Concussion | 2 | Military Concussion = 1,161 No concussion = 1,612 | 0.00 | 1.88 (1.43, 2.48) |
| Concussion | 3 | Athletes Concussion = 703 No concussion = 25,312 | 0.00 | 1.97 (1.47, 2.63) |

In athletes only, five studies reported an Odds Ratio including a total of 33,627 participants. This analysis revealed the Odds Ratio of athletes with a previous concussion sustaining a secondary concussion is 4.44 compared to athletes with no history of concussion. Three studies reported a rate ratio including a total of 26,015 participants. The pooled Rate Ratio for athletes with a previous concussion sustaining a secondary concussion is 1.97 compared to those with no previous concussion.

In service members only, two studies reported an odds ratio including a total of 2,773 participants. The pooled Odds Ratio for service members with a previous concussion sustaining a secondary concussion is 1.88 compared to those with no previous concussion.

Across all studies, three studies reported a hazard ratio for risk of extremity injury (upper or lower) in service members and athletes with a total of 537,157 participants (data not shown). The pooled Hazard Ratio is 1.61 for an extremity injury in those with a concussion compared to those with no previous concussion. In service members only, two studies reported on risk of upper and lower extremity injuries with a total of 536,937 participants. The pooled Hazard Ratio is 1.60 for an extremity injury for those with a concussion compared to those with no previous concussion.

Looking specifically at lower extremity injuries in military members and athletes, seven studies reported an Odds Ratio including a total of 44,999 participants. The summary estimate of those with a previous concussion sustaining a lower extremity injury is 1.60 compared to those with no previous concussion. Two studies reported a Hazard Ratio for service members and athletes with 23,264 participants. The pooled Hazard Ratio for lower extremity injury is 1.39 compared to those with no previous concussion.

Considering lower extremity injury in athletes only, six studies reported an Odds Ratio including a total of 21,955 participants. The Odds Ratio of those with a history of concussion sustaining a lower extremity injury is 1.82 compared to athletes with no history of a concussion. Two studies reported a rate ratio including a total of 164 participants. The pooled Rate Ratio for athletes with a previous concussion sustaining a lower extremity injury was 1.74 compared to those with no previous concussion.

Across the 17 meta-analyses completed, the results revealed significantly increased risk of all injuries; concussion; any extremity injury; and lower extremity injuries in service members and athletes with a history of concussion when compared to those with no history of concussion. This significant increase in risk is apparent when looking at odds of injury, time to event data (hazard), and rate of injury based on number of exposures.

The association between concussion and secondary injury may relate to residual physiological effects from the original concussion, mainly deficits in neurocognition and neuromotor control. This can be extended to include residual deficits in sensorimotor control. Immediate effects of concussion frequently include functional disturbances of the sensorimotor control system. Deficits in vestibular, visual, and proprioception senses, as well as postural and oculomotor control systems may be present after a concussion. In athletes, despite symptomatic recovery, medical clearance, and return-to-sport, sub-clinical deficits in sensorimotor function may remain long after an athlete is returned to the playing field.

Concussion may cause changes in postural control variability as measured by the Sensory Organization Test. Previously concussed but medically cleared athletes may also have impairment in dynamic stability during dual-task activities as compared to controls. There is thus evidence of persistent sensorimotor disturbances in those conventionally judged fit for return to normal activity. These sub-clinical deficits can negatively impact agility, coordination, balance, and reaction, leading to increased risk of injury.

Embodiments described herein allow for effective sensorimotor interventions after concussion, including during the acute phase of recovery. Individualized sensorimotor therapies provided by the described embodiments can be effective in providing recovery from the functional sensorimotor deficits associated with concussion. By training the sub-components of sensorimotor control, risk of secondary injury may be shifted down. Those who sustain a concussion may therefore significantly benefit from active intervention provided by the described embodiments and the associated improvements in sensorimotor and neuromotor deficits.

### Definitions

To assist in understanding the scope and content of this written description and the appended claims, a select few terms are defined directly below. Although these and other terms are employed herein, they are intended to be used in a for purposes of description and not for formal purposes of limitation. All terms, including technical and scientific terms, as used herein, have the same meaning as commonly understood by one of ordinary skill in the art to which this invention belongs unless a term has been otherwise defined. It will be further understood that terms, such as those defined in commonly used dictionaries, should be interpreted as having a meaning as commonly understood by a person having ordinary skill in the art. It will be further understood that such should be interpreted as having a meaning that is consistent with their meaning in the context of the relevant art and the present disclosure.

“Functional performance” means an individual’s composite use of multiple sensorimotor activities, based on the input of sensory information, to control a multi-joint or whole-body motor response to perform a specific task. A “functional performance metric” is a score or objective measure that indicates the user’s level of proficiency in performing in a specified area of functional performance. Examples of evaluations that may be utilized to determine a functional performance metric include any evaluation (in a virtual environment or in the real world environment) that scores one or more of balance, inspection time, agility, and/or coordination according to one or more standards such as accuracy, precision, strength, speed, or endurance, and in a manner that allows for objective comparisons between attempts.

“Sensorimotor Activity” means an element of sensorimotor control that primarily relies of one type of, or a limited set of, sensory information to control a localized motor response. Sensorimotor activities include, but are not limited to, smooth pursuit, saccades, near-point convergence, peripheral vision, cervical joint proprioception, and kinesthetic awareness. A “sensorimotor activity metric” is a score or objective measure that indicates the user’s level of proficiency in performing the sensorimotor activity.

“Sensorimotor control” means the global ability of an individual to receive sensory information and use the sensory information along with cognitive processes to complete skilled motor actions, including sensorimotor activities.

“Sensory information” and “sensory stimuli” refer to information collected through one or more of visual, vestibular, or joint/muscle mechanoreceptor inputs. Once the information is received, the central nervous system of the individual processes and integrates these inputs, which may occur fully or partially subconsciously, to direct movement.

“Virtual Immersive Environment” refers to a virtual, three-dimensional environment displayed to the user by way of an HMD and/or other display device. Although a virtual immersive environment may be illustrated herein using two-dimensional figures and related description, it will be understood that the virtual immersive environment will provide the an actual three-dimensional environment to the user.

### Computer System Overview

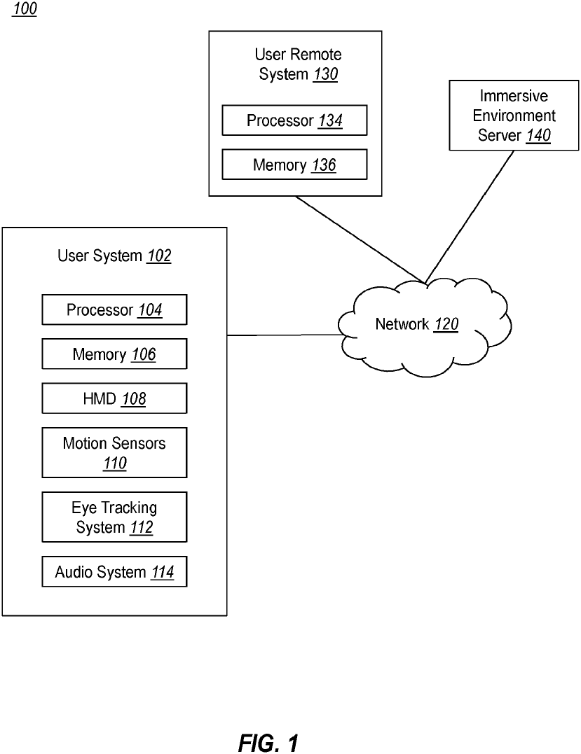

FIG. 1 illustrates an exemplary computing environment 100 configured for providing a virtual immersive environment for improving sensorimotor control and functional performance. The illustrated computing environment 100 may also be referred to herein as an Immersive Visual-Vestibular Interface Sensorimotor System (IVVISS) and/or as a Virtual Immersive Sensorimotor Training (VIST) system. Although many of the examples described herein will be made in reference to treatment of a neurological impairment associated with concussion injuries, other applications include the treatment of neurological impairment associated with aging and/or other injuries, prevention of neurological impairment, improvement of neurological response to sensory stimuli, or “super-training” individuals that do not necessarily have a pre-existing injury or impairment.

As shown, the computing environment 100 includes a user system 102. The computing environment 100 may also optionally include a user remote system 130 and/or immersive environment server 140 connected to the user system 102 and/or to each other by network 120. The network 120 may represent one or more of a cellular network, Local Area Network (“LAN”), a Wide Area Network (“WAN”), or the Internet, for example. The network 120 may thus include wireless and/or wired connections between the various components of the computing environment 100. Physical connection interfaces include USB, HDMI, DVI, VGA, fiber optics, DisplayPort, Lightning connectors, Ethernet, and the like.

The illustrated user system 102 includes one or more processors 104 and memory 106 (e.g., in the form of one or more hardware storage devices). Types of processors include microprocessor, video processors, application specific integrated circuits (ASICs), and systems on a chip (SOACs). Types of memory include RAM, ROM, DRAM, SRAM and MRAM, which may be stored on a hardware storage device such as disk media, electronic, or other like bulk, long-term storage or high capacity storage medium. Types of firmware include static data or fixed instructions, BIOS, system functions configuration data or other routines used for operation.

The user system 102 also includes a head-mounted display (HMD) 108 configured to display a virtual immersive environment to the user when worn by the user. The term HMD should be understood to be synonymous with similar terms referring to similar display devices such as “headset,” “VR device,” “VR display,” and the like.

The user system 102 may also include one or more motion sensors 110, including sensors for measuring the position and/or movement of the head, hand, and/or other body parts. Particular examples of motion sensors 110 include accelerometers (e.g., to detect reaction movement of the head and/or hand to stimuli), gyroscopes (e.g., to measure the position of the head and/or hand), and magnetometers (e.g., to measure physical orientation of the user).

Motion sensors 110 associated with the hand may also be utilized to allow the user to select objectives, navigate in-display menus, make in-display selections, and interact with one or more virtual objects displayed within the virtual immersive environment. Collision detection (i.e., detecting the intersection of one or more objects or object paths) between the user’s hand and one or more virtual objects may also be determined by way of motion sensor(s) 110 of the hand.

Although most of the examples described herein relate specifically to measuring head and hand movement, it will be understood that the same concepts may be readily applied to other body parts, and sensorimotor activities that relate to other body parts in addition to or in contrast to head and hand movement are also included within this disclosure.

The user system 102 may also include an eye tracking system 112 for measuring eye position and/or movement of one or both eyes in response to sensory stimuli, including visual and/or vestibular stimuli. The user system 102 may also include an audio system 114, which may include one or more speakers for communicating audio to the user and/or one or more microphones for receiving audio from the user.

As described in more detail below, the user system 102 operates (e.g., by running appropriate software) to present a user with a virtual immersive environment where sensory stimuli may be provided in a controlled manner and where one or more sensorimotor activities may be performed. The user system 102 can operate to provide analysis of the user’s motor and/or visual responses to the sensory stimuli by way of the motion sensors 110 and/or eye tracking system 112. For example, as the user moves in response to the sensorimotor activities performed within the virtual immersive environment, the associated movement data is collected and analyzed for parameters such as accuracy, stability, precision, timing, coordination, and the like.

Eye tracking may be utilized to measure the location of the user’s gaze, the speed, accuracy, latency, or smoothness of the user’s eye movements, or combination thereof. Eye tracking may also be used to count the number of blinks and measure the pupil size in during the activity. The eye tracking system 112 may include a digital camera that enables the capture of eye, and specifically pupil, movement in response to stimuli. The digital camera preferably operates at a frame rate of at least 120 Hz and may capture the eye focus of the user at approximately the same rate for recording by the user system 102 and/or the remote user system 130.

An example of an eye tracking device that may be utilized in the disclosed user system 102 is the commercially available system by HTC with the trade name Vive Pro Eye that contains Tobii eye tracking. In common use, such systems track eye movement to a region being viewed for the purpose of limiting the content of the 3D information displayed to the user in order to permit faster response time to the viewer of the 3D environment. In contrast, the user system 102 utilizes such eye tracking systems to measure the aforementioned parameters of eye movement, including in some cases the ability to find a specific target, and the reaction time to find object.

The user remote system 130 is a computer device separate from the user system 102, and includes its own processor(s) 134 and memory 136. The user remote system 130 may be utilized to process the inputs and outputs of the virtual immersive environment displayed by the user system 102. For example, the user remote system 130 may have greater computer processing power than the onboard processor(s) 104 of the user system 102, and the processing required to run the virtual immersive environment and/or analyze user responses may be divided among the user system 102 and user remote system 130 according to the needs of the particular hardware at hand. The user remote device 130 may be a mobile computer device, such as a mobile telephone, tablet, or the like, a desktop personal computer, or other suitable computer device.

The immersive environment server 140 may also be utilized to contribute to the computer processing required to run the virtual immersive environment. Additionally, or alternatively, the immersive environment server 140 can connect to other remote computer devices, such as those associated with healthcare providers, trainers, coaches, and the like. This allows these remote devices to send instructions to the user remote system 130 and/or user system 102 to control the virtual immersive environment and to receive results according to the user’s interaction with the virtual immersive environment. For example, a healthcare provider may be able to remotely select or adjust a training/therapy regimen (e.g., adjust type of activities and/or difficulty) within the virtual immersive environment, monitor the user’s execution of the associated sensorimotor activities in real time, adjust instructions on the fly, and provide remote, real time assessments and/or feedback.

### Methods for Training Sensorimotor Control

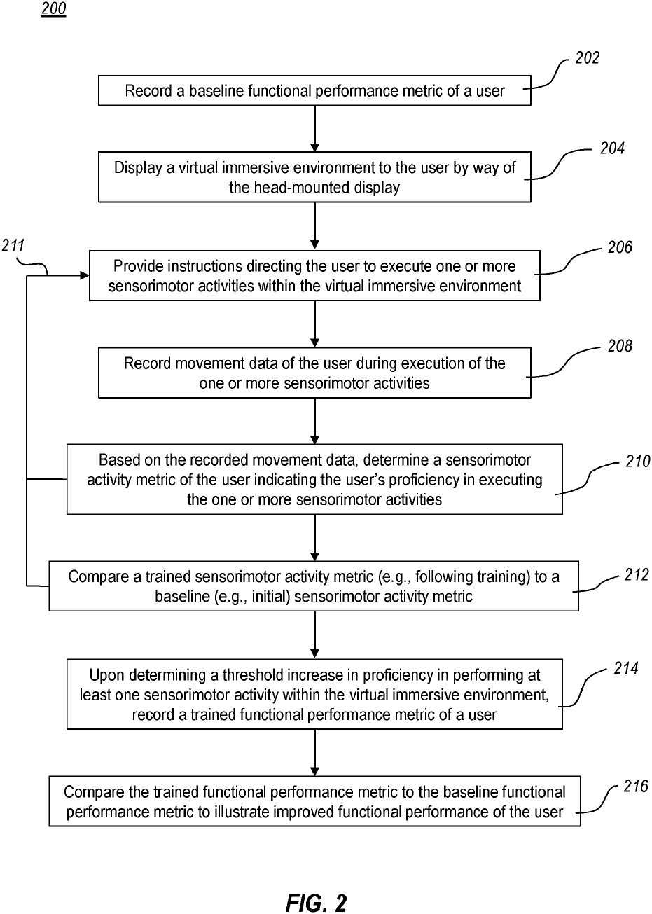

FIG. 2 illustrates a method 200 for training sensorimotor control to enhance functional performance, reverse or slow neurological impairment, and/or prevent injury in a user. The illustrated method may be at least partially implemented using the computer systems described above in relation to FIG. 1.

In the method 200, a baseline functional performance metric of a user is recorded (step 202). The functional performance metric may be related to, for example, balance, reaction time, agility, and/or coordination. The baseline functional performance metric may be obtained via an evaluation outside of the virtual immersive environment, such as through a conventional clinical evaluation. Alternatively, and more preferably, the baseline functional performance metric may be obtained via an evaluation within virtual immersive environment, where more objective metrics may be obtained. Examples of specific functional performance evaluations (e.g., balance, inspection time, visual figure-ground) that utilize the virtual immersive environment are described below. In some embodiments, a physical therapist, other healthcare provider, trainer, or the like involved in the evaluation may associate with the user remotely.

The computer system may display a virtual immersive environment to the user by way of the HMD (step 204). The computer system also provides instructions (e.g., audio, visual, or both) to the user directing the user to execute one or more sensorimotor activities within the virtual immersive environment (step 206). The instructions may also include instructions for positioning the user’s body, feet and/or hands in specific positions such as particular stances for balancing on the floor, or other structures in the real environment. As described above, these instructions may be controlled from a remote computer system associated with a physical therapist, other healthcare provider, trainer, or the like. The method 200 therefore beneficially allows remote training and/or evaluating of patients/users, as well as real-time feedback and control from the remote person(s).

The computer system then records movement data of the user associated with user movements during execution of the one or more sensorimotor activities (step 208). The movement data may be captured using the various motion sensors and/or eye tracking systems described above, and may relate to position, change of position, and speed of movement of various body parts including the head (e.g., 3D orientation of the head as represented by axes of pitch, roll, and yaw), hand(s), and eyes, for example.

Based on the recorded movement data, the computer system determines a sensorimotor activity metric of the user indicating the user’s proficiency in executing the one or more sensorimotor activities (step 210). This may be transmitted remotely to the system associated with a physical therapist, other healthcare provider, trainer, or the like.

As indicated by arrow 211, the process of providing sensorimotor activity instructions, recording corresponding user movements, and determining the sensorimotor activity metric (steps 206, 208, and 210) may be repeated a number of times. A sensorimotor activity metric may be reported back to the user visually and/or by audio as an indication of improvement relative to a “normal” response and/or to previous individual training evaluations. The normal response may be predicted or calculated as an average of typical, healthy users or as a score typical of user’s under similar circumstances and/or demographics.

Following two or more sets of sensorimotor activities, the computer system may then compare a trained sensorimotor activity metric (e.g., following training) to a baseline (e.g., initial) sensorimotor activity metric (step 212). If the trained sensorimotor activity metric has not yet reached a predetermined threshold level of improvement over the baseline sensorimotor activity metric, the computer system may repeat steps 206, 208, 210 before repeating step 212. In some embodiments, if the sensorimotor activity metric falls outside a predetermined normal range, the difficulty of the sensorimotor activities may be automatically adjusted accordingly. A physical therapist, other healthcare provider, trainer, or the like may also control/adjust the selection of training activities and/or their difficulty. Again, the physical therapist, other healthcare provider, trainer, or the like may do this remotely.

Upon determining a threshold increase in proficiency in performing at least one sensorimotor activity within the virtual immersive environment, the computer system may then record a trained functional performance metric of a user (step 214). The trained functional performance metric is preferably obtained in similar fashion to the baseline functional performance metric to enable objective, direct comparisons between the two. The method then compares the trained functional performance metric to the baseline functional performance metric to illustrate improved functional performance of the user (step 216).

Performance of the method beneficially enables improved clinical outcomes as measured by improved functional performance. The benefits of the described method can apply to those that have neurological impairment associated with injuries, including concussion injuries, aging, or other causes of neurological impairment. The described method can also benefit those that do not necessarily have neurological impairment, but nonetheless can see increases in functional performance. The described method can also beneficially train users in a manner that makes them less likely to suffer injury.

### Exemplary Sensorimotor Activities for Training Within a Virtual Immersive Environment

In traditional clinical practice, it is very difficult to capture a person’s visual attention for a long enough period of time to effectively train elements of sensorimotor control. The virtual immersive environment described herein beneficially captures the entirety of the user’s visual field and thus their visual attention, and therefore permits training for an extended and more focused period of time. Additionally, the virtual immersive environment permits greater variability and control in the speed and positioning of target objects for eye movement training or other sensorimotor activities, greater variability in the visual environment, and the controlled use of visual distractions to more effectively control difficulty of the exercises.

Moreover, as mentioned above, a remote healthcare provider or trainer may use a remote computer device to monitor and control the virtual immersive environment and the sensorimotor activities presented to the user, including controlling the variability of speed, size, shape, color, movement, or number of target objects, for example. The remote healthcare provider or trainer may also make adjustments in real time and provide feedback and/or assessments of the user’s activities.

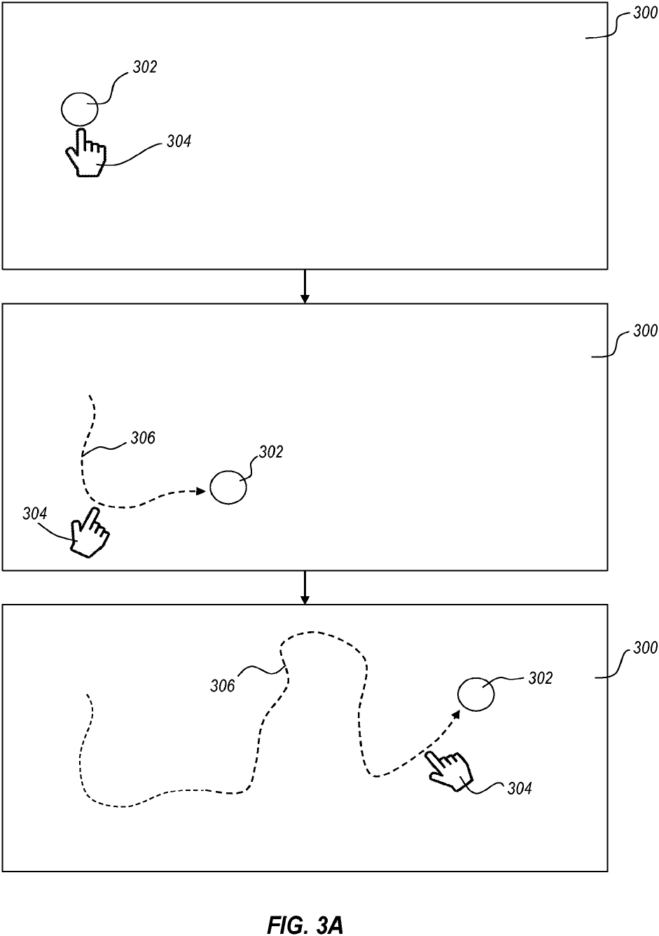

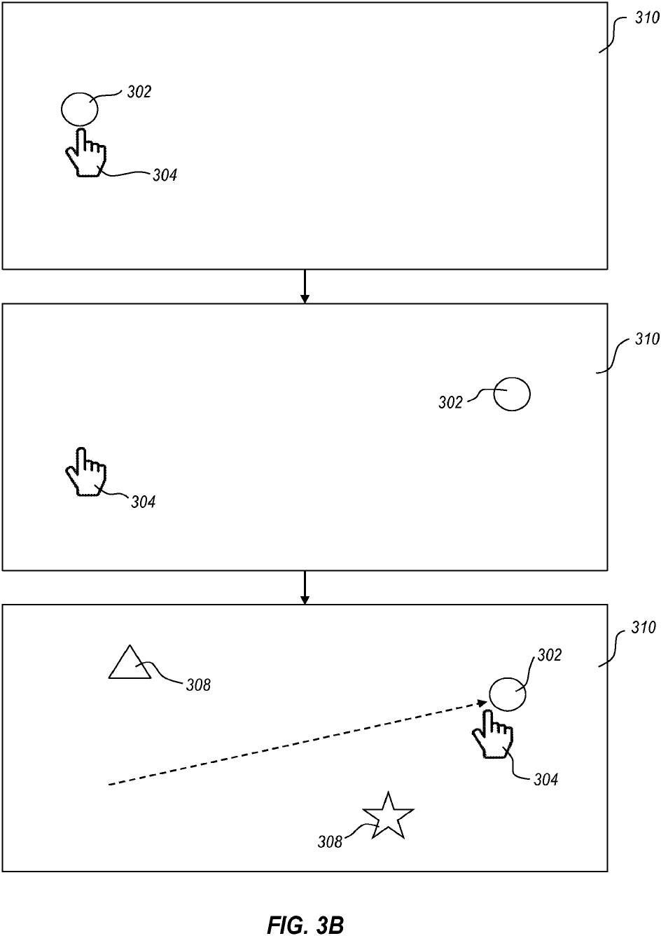

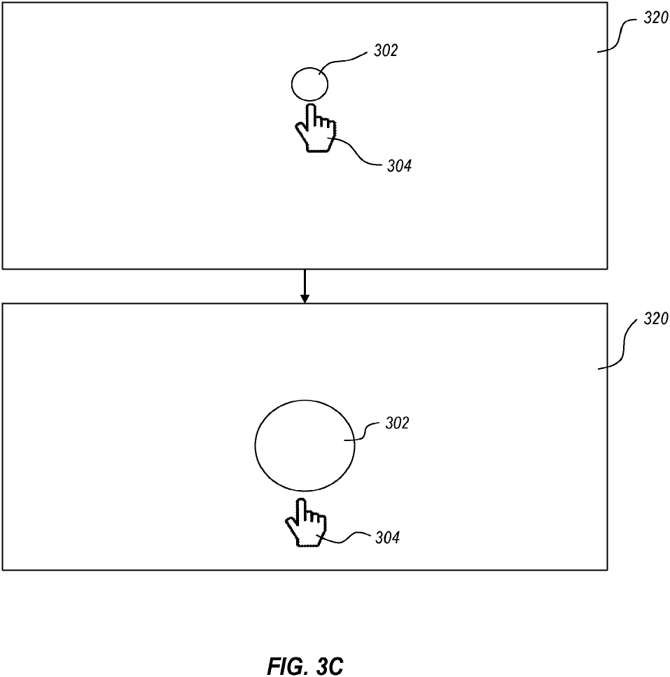

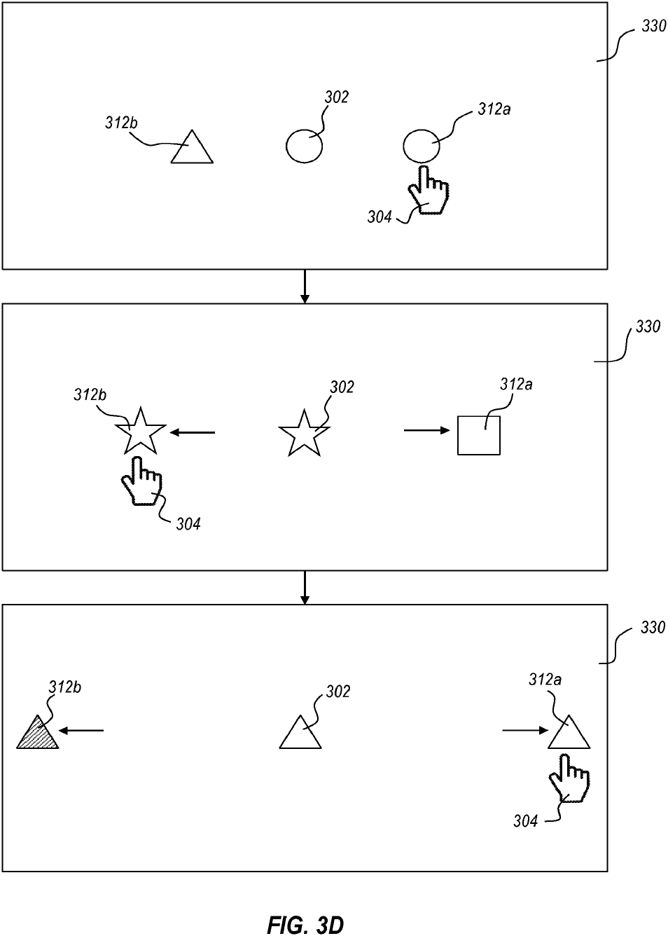

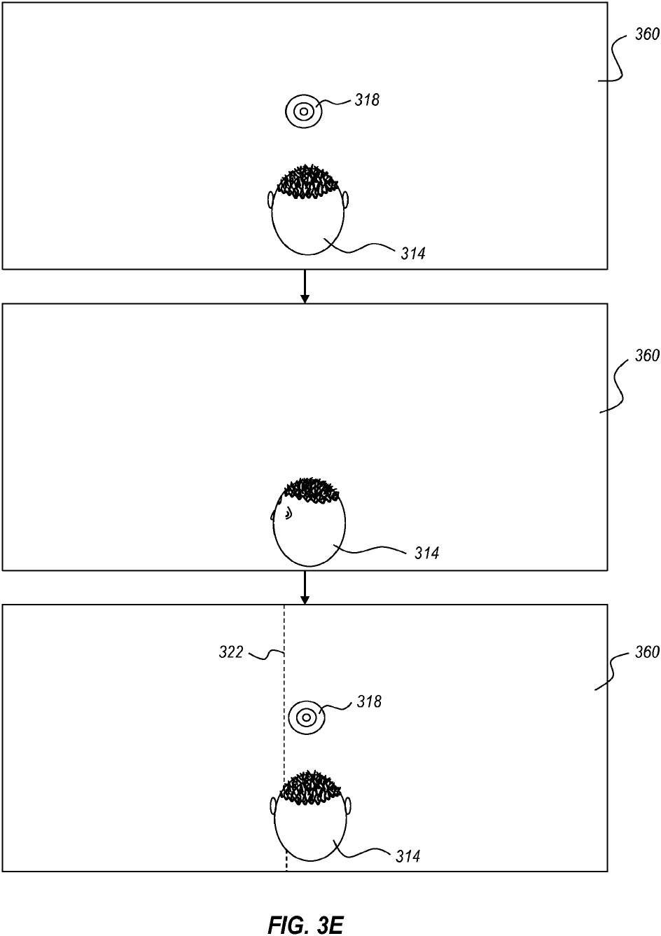

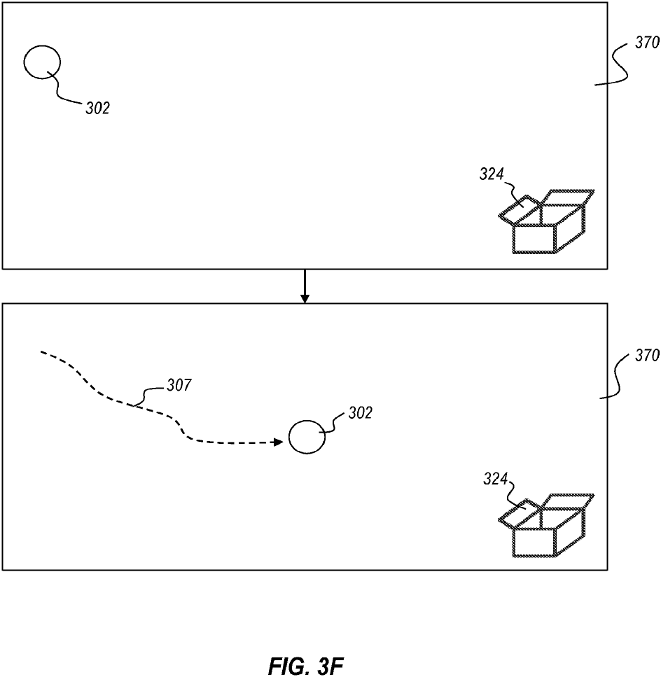

FIGS. 3A through 3H illustrate some exemplary sensorimotor activities that may be performed by the user within the virtual immersive environment, and that may be utilized to improve functional performance of the user. Any of the below examples may be modified by requiring one or more additional movements from the user during execution of the sensorimotor activity, thereby changing the activity to a functional performance activity. For example, the user may be instructed to perform the sensorimotor activity while positioning and/or moving his/her head in a specified manner (in addition to what is already required by the activity), thereby activating the vestibular system and cervical mechanoreceptors.

### Smooth Pursuits

FIG. 3A illustrates an example of a virtual immersive environment 300 configured to provide a “smooth pursuit” sensorimotor activity, and how the virtual immersive environment 300 may change over time during performance of the activity. The smooth pursuits activity can be utilized to train accuracy and speed of smooth pursuit binocular eye movements. A target object 302 positioned in the virtual immersive environment 300 can move unpredictably (in all directions including towards and away from the individual), and with varying speeds. The user is directed to follow the path of the moving object while tracing the object (as shown by trace line 306) using a manual pointer 304 associated with a hand control. The trace line 306 need not necessarily be displayed in the virtual immersive environment 300 but is shown here for illustrative purposes.

At the easier settings, the object 302 will move in a smaller range, more slowly, and within a less distracting environment. Progressive levels will adjust one or more difficulty parameters such as by increasing the range of movement of the object 302, thus requiring both head and eye movement to follow the object 302, by causing the object 302 to move more quickly and/or with changing speeds, and by providing the object 302 in a more distracting and visually stimulating environment. The sensorimotor activity metric may be presented to the user as a score based on accuracy of manually tracing the path of the target object 302. For example, the greater the amount of collision between the actual path traversed by the object 302 and the path traced by the manual pointer 304, the better the resulting metric/score.

### Saccades

FIG. 3B illustrates an example of a virtual immersive environment 310 configured to provide a “saccades” sensorimotor activity, and how the virtual immersive environment 310 may change over time during performance of the activity. The saccades activity may be used to train accuracy of pro-saccadic and anti-saccadic eye movements, along with selective attention. A target object 302 will appear within the virtual immersive environment 310, and the user will be directed to move their eyes toward the object 302 and utilize the hand controller to select the location of the object with the pointer 304. The object will then disappear and appear at some other location within the virtual immersive environment 310, at which point the user should repeat the task of locating and pointing to the object.

At the easier levels, the object 302 will only appear in a range that does not require head movement and the user will complete a pro-saccade. Progressive levels will increase the range of movement required between the saccadic movements, including requiring both head and eye movement to see the object 302. Other difficulty parameters may additionally or alternatively be adjusted to cause the object 302 to move more quickly, with changing speeds, in a more distracting environment, or combination thereof. For example, distraction objects 308 may begin to appear within the virtual immersive environment along with the target object 302.

The saccades activity may also be configured to train anti-saccadic movement. Anti-saccadic movement may be trained in like manner except that the user will be directed to move the eyes away from the target object 302, in the opposite direction, thus requiring increased cognitive control and attentional focus. The sensorimotor activity metric may be presented to the user as a score based on accuracy of selection of the object with the pointer 304 from the hand controller. Reaction time may also be built into the score.

### Near-Point Convergence

FIG. 3C illustrates an example of a virtual immersive environment 320 configured to provide a “near-point convergence” sensorimotor activity, and how the virtual immersive environment 320 may change over time during performance of the activity. The near-point convergence activity may be utilized to improve the user’s point of near binocular visual convergence. The user is directed to follow the movement of a target object 302 as it moves towards and away from the eyes in the virtual environment 320. This can be done as a continuous smooth movement of the object 302 or by a rapid relocation of the object (near and far). The subject will be asked to move their eyes to the location of the object and use the hand controller and pointer 304 to indicate position of the eyes.

Progressive levels of difficulty may adjust one or more difficulty parameters such as making the movement of the object variable in speed, requiring faster selection, increasing the complexity of the visual environment, or combinations thereof. The sensorimotor activity metric may be presented to the user as a score based on accuracy of selection of the object 302 with the pointer 304 as controlled by the hand controller. Reaction time may also be built into the score.

### Peripheral Vision Acuity

FIG. 3D illustrates an example of a virtual immersive environment 330 configured to provide a “peripheral vision acuity” sensorimotor activity, and how the virtual immersive environment 330 may change over time during performance of the activity. A target object 302 is displayed in the center of the field of view, and a series of side objects 312a and 312b are presented on each side. In preferred embodiments, multiple side objects may be presented on either side of the central target object 302, such as in stacked columns of multiple objects (e.g., about 3 to 5). Here, a single side object is shown on either side of the central target for convenience. The user will use their peripheral vision to identify the side object 312a or 312b that corresponds to the central object 302, and will be directed to use the hand controller to select the correct side object 312a or 312b with the pointer 304.

The appearance (e.g., color, shading, and/or shape) of the central object 302 may change in response to a manual activation by the user, in response to selection of a side object 312a or 312b, or at a predetermined interval of time. Additionally, the side objects 312a and 312b may move towards the periphery as the activity progresses (e.g., with each change of the central object 302).

One or more difficulty parameters may be adjusted by changing the speed at which the central object 302 changes appearance, constant vs. changing appearances of the side objects 312a/312b, the complexity of the visual environment, or combination thereof. The sensorimotor activity metric may be presented to the user as a score based on the number of correct indications within a given timeframe. Additionally, eye tracking may be used to determine the degree to which peripheral vision is being used to identify correct side objects 312a/312b rather than saccadic eye movement.

### Cervical Joint Proprioception & Kinesthetic Awareness

FIG. 3E illustrates a virtual immersive environment 360 configured to provide a “joint-position error” sensorimotor activity, and how the virtual immersive environment 360 may change over time during performance of the activity. The joint-position error activity may be utilized to train the user’s cervical neuromotor and proprioceptive control. A target 318 will be displayed to the user within the virtual environment while the user’s head 314 is in midline. A pointer may be displayed to indicate to the user when the head is directly aligned with the target 318. Then the screen will go black or otherwise be disrupted, and the user will be instructed to actively move their head to a new location (e.g. left, right, up or down) and then to return their head back to the original midline position without the use of visual information.

When the user believes he/she has positioned the head 314 at midline again, a manual selection may be made and the screen will illuminate or otherwise revert to the original scene to indicate how far off midline the subject’s head is from the original starting position, such as via indication line 322. Progressive levels of difficulty may be incorporated by varying the starting position (i.e. midline to non-midline) and/or the range of motion required to move the head 314 away from the target 318 before relocation to the original position. The motion sensors of the HMD (e.g., external headset trackers, accelerometers, and gyroscopes) are used to measure the degree of error relative to the starting position after each movement (as recorded by the position of the head 314 at the time the manual selection as made by the subject) and this is used to calculate a sensorimotor activity metric.

### Cervical Neuromotor Control

FIG. 3F illustrates a virtual immersive environment 370 configured to provide a “cervical neuromotor control” sensorimotor activity, and how the virtual immersive environment 370 may change over time during performance of the activity. The user is instructed to move his/her head to control to movement of an object 302 displayed in the virtual environment. For example, the user may be tasked with moving the object 302 to a target location 324. Head movements measured by the user device correspond to path 307 traversed by the object 302. A related activity involves moving the head to control movement of an object through a maze.

The sensorimotor activity metric may be based on speed and the ability to maintain correct position vertical of the device (e.g., a head-neck position of neutral postural alignment as opposed to capital extension). Progressive levels of difficulty may be incorporated by varying the difficulty of the action goal and therefore the neuromotor control required to successful complete the activity, for example. The motion sensors of the HMD (e.g., the external headset trackers, accelerometers, and gyroscopes) may be used to measure the degree of capital extension relative to the starting position.

### Evaluation of Users Pre- and Post-Sensorimotor Training for Improvements in Functional Performance

Evaluations may be utilized to determine the extent by which individual sensorimotor activities contribute to overall sensorimotor control. Each discrete activity can advance a type of functional performance desired by providing additional inputs of sensory information, utilizing a more distracting and visually stimulating environment, and changing the speed and amount of required motor response.

Functional performance evaluations include, but are not limited to, balance, inspection time, coordination, and agility. These, and other types of functional performance evaluations, require the efficient engagement of multiple senses, cognitive processes including decision making, and/or complex motor responses. These evaluations may be performed in a traditional clinical setting but are difficult to precisely measure. Functional performance evaluation can be modified for the virtual immersive environment to provide more precise metrics of success. The described embodiments also provide additional benefits such as the ability for remote evaluation, which reduces time and cost.

Below are a few exemplary evaluations that may be utilized to measure functional performance in a user and that may be utilized to determine whether training with one or more sensorimotor activities has resulted in an improvement in functional performance. In addition to the examples listed below, any of the above sensorimotor activity examples may be modified by requiring one or more additional movements from the user during execution of the sensorimotor activity, thereby changing the activity to a functional performance evaluation. For example, the user may be instructed to perform the sensorimotor activity while positioning and/or moving his/her head in a specified manner (in addition to what is already required by the activity), thereby activating the vestibular system and cervical mechanoreceptors.

### Balance

Human control of balance is frequently measured in traditional clinical practice. This measurement is a global assessment of postural stability and is dependent upon overall sensorimotor control, with vision, vestibular and mechanoreceptor sensory integration. Functional performance relies upon two or more sensorimotor components. There are many outcome measures, including the Timed-Up-and Go test, the Berg Balance Scale, and others that are completed with pen and paper. Expensive technologies have been developed to increase the sensitivity and precision of balance measurements, including the Neurocom Balance Master and other versions of force plate sensors. More recently, mobile devices have been utilized to provide inexpensive yet highly sensitive, reliable and valid measurements of balance. Each of these methods has established metrics of normative values for various age-groups and/or cut-points of performance which are predictive of fall or injury risk.

In some embodiments, the Sway Balance App, commercially available from Sway Medical, was used pre- and post-training. Each individual’s balance is measured using the triaxial accelerometer in a smartphone while the individual stands in 5 stance positions: feet together, tandem with right foot in front, tandem with left foot in front, and single leg stance (left and right). Evaluations are completed with the eyes closed. A score is derived for each standing position as well as an overall balance score on a 0-100 point scale, with higher scores indicating better balance.

In another embodiment, postural control and balance may be evaluated in a visually challenging environment by displaying a moving visual environment to the user. This may include inaccurate visual information (i.e., that does not correspond to the real-world) such as having the horizon shift (tilt) left and right. This will require the subject to rely on vestibular and proprioceptive information to maintain postural stability. Motion sensors such as headset accelerometers and gyroscopes are utilized to measure the amount of body sway, with the head in midline, while observing this challenging visual environment. Adjustments may be incorporated by increasing degree and speed of environmental tilt. Evaluation scores may be based off total amount of measured postural sway.

### Inspection Time

Inspection time is a type of reaction time. Inspection time is strongly linked to processing speed, requiring visual attention, discrimination, perception, cognition, and a simple motor response. In traditional clinical practice, inspection time is not routinely trained or evaluated. Visual perception and cognitive processing are, however, skills that humans use every day.

A measurement of inspection time may be accomplished through the use of a test administered on a smartphone, such as by using the Sway Medical Application. The subject is presented with visual instructions regarding under what circumstances and how to move the device to respond to the visual stimulus. Here, the individuals are presented with 2 lines, one on the left and one on the right side of the horizontally held screen. The individual rapidly moves the phone to the right if the right line is perceived to be longer and to the left if the left line is perceived to be longer. The test may progress in difficulty, with the lines becoming more alike in size. The application measures time to respond and accuracy of performance when the accelerometer in the phone detects the direction of movement. Scores may be based on a 0-100 point scale, with higher scores indicating better performance.

In another embodiment that may utilize a virtual environment, the user is presented with verbal or visual instructions regarding under what circumstances to move the head or select an object with the hand controller. This may include change in screen color, object proximity to the eyes, color of the object, size of the object, other visual changes to the display, and combination thereof. Progressive levels of difficulty may be incorporated by changing the type of reaction time being trained and/or the requirement of the subject to process additional information or distracting information during the completion of the evaluation. Motion sensors such as headset accelerometers and gyroscopes and/or hand motion sensors (e.g., in the hand controller) are used to measure movement time in response to a target stimulus.

### Visual Figure-Ground

The visual figure-ground activity evaluates an individual’s scanning vision, visual selective attention, and discrimination. The user is presented with an object that they must locate within a visually complex environment and select using a manual activation. Various oculomotor strategies (smooth pursuits, saccades, convergence) combined with selective attention, visual discrimination, perception, and cognition, are required to successfully complete this evaluation. Once the object is identified by the individual, it is selected using a pointer from the hand controller to collide with the object in the virtual environment. After each selection, the environment will refresh, moving the object to a new location within the reset environment. The use of the manual selection to quantify successes through a derived metric/score provides objectivity to the evaluation and the ability to monitor progress across time.

The user’s ability to visually inspect and attend to an object within the environment using various oculomotor strategies (smooth pursuits, saccades, convergence) contributes to the metric/score. The metric/score may be based on number of correct identifications within a specified time, for example.

### Coordination

Coordination is the ability to switch between subtasks or activities in order to complete a complex performance skill. This requires sensory information processing from all sensory sources, including feedforward to plan a movement and feedback to alter the planned movement as needed in order to succeed with the intended action. Quick motoric response is also required to shift posture, maintain balance, and use the upper and lower extremities symmetrically or asymmetrically.

One evaluation of this type of functional performance is hand-eye-coordination. Hand-eye coordination can be tested in different manners for different populations. In a preferred example, the individual stands 2 meters from a wall with a tennis ball. The individual throws the ball to the wall with their right hand and catches it with the left. This action is repeated with the throw from the left to a catch with the right. The individual is instructed to complete as many throw-and-catch sequences as possible in a given time period (e.g., 30 seconds). In an alternate test of eye-foot coordination, this same sequence can be completed with a soccer ball and a kick from the left foot, receiving with the right and vice-versa for a given time period. The individual’s performance prior to sensorimotor training can be compared to their performance after training.

### Agility

Agility is perhaps the most complex type of functional performance ability. Agility combines balance, speed, coordination, and power to complete complex actions incorporating two or sensorimotor control components. Typically, tests of agility are reserved for the athletic or military population and are not routinely completed in older adults, however related tests of functional performance, including functional mobility, may be used to evaluate an older adult.

One test performance of agility with multi-directional changes is the Lateral Change of Direction Test. In this test, three cones are set up on a straight line, one in the middle and one on the right and left, each 5 meters (or some other fixed distance) from the middle cone. The individual begins standing and facing the middle cone. An examiner directs the individual to “go” and indicates to move left or right. The individual runs in the direction instructed, touches the cone and runs to the cone on the opposite side, touches it and returns to the middle cone. A stopwatch is used to time from “go” until the middle cone is touched.

### EXAMPLES

### Example 1: Experimental Study Design to Assess Sensorimotor Activity Training for Improving Functional Performance in Collegiate Soccer Athletes

The purpose of this study was to assess the use of sensorimotor control training utilizing sensorimotor activities delivered through immersive virtual environments between a test group and a control group by quantifying 1) the training effect measured via improvement pre- to post-intervention on the immersive virtual sensorimotor activities and 2) the difference in the in immersive virtual sensorimotor activity proficiencies and clinical measures of functional performance.

Trial Design: This study was conducted as a quasi-experimental design with two participating institutions. Baseline performance was collected at the beginning of the study on each immersive virtual sensorimotor activity exercise and on the clinical tests of sensorimotor control for all participants. These measures were repeated 6 weeks later after training. This research protocol was registered prospectively on clinicaltrials.gov and there were no changes to the methods after trial commencement. The study was approved by the University of Mississippi Medical Center Institutional Review Board (IRB), the University of West Alabama and Mississippi College IRBs prior to initiation. Informed written consent was received prospectively and the rights of each participant was protected.

Participants: Participants for the test group of the study were recruited from the men’s and women’s soccer teams at Mississippi College (MC) in Clinton, MS, USA. Participants for the control group of the study were recruited from the men and women’s soccer teams at The University of West Alabama (UWA) in Livingston, AL, USA. Both institutions are Division II members of the Gulf South Coast Conference and the National Collegiate Athletic Association (NCAA). Recruiting was completed when the athletes reported to campus for the 2019 season. Soccer was chosen as the sport of interest because of dual representation of both sexes and because of equivalent rates of injury (8.07 and 8.44 per 1000 exposures). Athletes eligible to play in the 2018 soccer season, aged 18 or over, were eligible for inclusion. Athletes with a current diagnosis of concussion (i.e. non-medically cleared), lower-extremity musculoskeletal injury, or other diagnosis that would prevent the athlete from participating in the intervention were excluded. Athletes with a seizure disorder and photo-sensitivity were excluded.

Sensorimotor Activity Training: The training delivered to the participants in the experimental group was 6-weeks long, consisting of two face-to-face sessions per week for each athlete in the experimental arm. All sessions were conducted in the strength and conditioning room at Mississippi College. Within the training, all participants completed 9 different training activities in an immersive virtual environment using the VR HMD by Oculus Quest. The sensorimotor training activities were aimed at the various sub-systems which contribute to overall sensorimotor control and functional performance. This included vestibular, visual and oculomotor activities, cervical neuromotor control training, coordination, and postural/balance exercises (Table 3).

TABLE 3

Sensorimotor Training Targeted with Immersive Virtual Exercises

| | | |
|---|---|---|
| Category | Sensorimotor Abilities | Functional Performance |
| Oculomotor control | Smooth pursuit | Figure ground discrimination |
| | Saccades | |
| | Convergence | |
| Visual Acuity | Peripheral vision perception | |
| Cervical Movement | Proprioceptive accuracy | Maze |
| Coordination | | Head-Eye coordination Eye-hand coordination |
| Postural Control | | Standing balance |

Each training sensorimotor activity was developed with 3 levels of progressive difficulty across time. Difficulty was progressed by increasing the speed, accuracy, range of motion, or coordination of the movement required to be successful. The virtual visual environment was also manipulated to progress the complexity of the exercises. At each session, the trainer chose 3-4 of the immersive virtual training exercises for that day. Each training exercise lasted 1-1.5 minutes in duration and were repeated in series for the duration of the 15-minute session. The completion of each exercise at each session resulted in a proficiency score, which was presented to the user in the virtual environment and represented a metric of their proficiency in executing the exercise. The control group completed no sensorimotor training in the immersive virtual environment.

Testing Methods and Outcomes: Each of the immersive virtual training activities included a testing scale. This permitted completion of the core sensorimotor activity proficiency or functional performance measure that was exactly the same for both testing groups. For example, for smooth pursuit, the moving object moved along the exact same spline within the graphic environment, at the same speed for both measurement time-points. Each immersive virtual activity produced a score which ranged in value between 0 - 1000, with higher scores indicating increased proficiency.

A description of the clinical measures of sensorimotor control used in this project are below. These outcomes were chosen because they have been extensively used in research related to concussion to measure various domains of sensorimotor control and have demonstrated clinical usefulness and psychomotor soundness.

Static Balance and Inspection Time was measured as follows. The Sway Balance App is a Food and Drug Administration approved medical device with research literature supporting its use as a valid and reliable baseline assessment of static balance in athletes. Sway uses the triaxial accelerometer in a smartphone and a proprietary algorithm to calculate postural sway and reaction time on a 0 - 100 point scale, where 100 is perfect. Participants completed this test by logging in to the Sway App on their phone. The balance score, includes measurement of postural sway in 5 standing positions for 10 seconds, all with the eyes closed. The total score is calculated across three separate trials in each of the 5 standing position. Inspection time was used as a more indicative determination of functional performance and complex measurement of reaction time. This test requires the participant to determine which line, presented on the left or right side of the screen is longer. This requires visual inspection, cognitive selection, and response through a movement of the device (right or left).

Deep Neck-Flexor Endurance Test (DNFET) was measured as follows. The participant was instructed to maximally “tuck the chin” and lift his/her head 1″ (2.5 cm) off the plinth table and hold. While in this position, the tester observed for the approximation of skin folds under the chin. The tester slid the widths of stacked index and middle fingers under the subject’s head at the most posterior aspect of the occiput so that the back of the subject’s head maintained tactile contact with the tester’s stacked fingers. During the test, the examiner provided a tactile cue for maintaining proper head position. Time recording began when the subject raised his/her head off the table and was terminated when 1 of 4 criteria was met where upon: 1.The edges of the skin folds no longer approximated because of a loss of chin tuck, 2.The subject’s head rested on the tester’s folded fingers for more than 1 second, 3.The subject raised his/her head above 1″ such that there was no longer contact with the tester’s fingers, or 4.The subject was unwilling to continue. The subject was only allowed one deviation from the test position, corrected by providing a verbal cue to direct him/her to resume the proper position and continue the trial.

Cranial Cervical Flexion Test (CCFT3) was measured as follows. CCFT3 was used as a test of cervical flexor control and endurance by having the participant recruit the deep neck flexors in a precise manner according to the Stabilizer biofeedback device (Chattanooga). As described by Jull, this test had 2 stages. Stage 1 (CCFT3): was used to measure the ability to recruit and control the deep cervical flexors. The highest level (22, 24, 26, 28 or 30 mmHg) that the participant could achieve and hold for 3 seconds with the correct muscle action, without palpable activity of the superficial flexors was recorded.

Statistical Methods: To compare demographic and injury history of the groups at baseline, t-tests for continuous, chi-square tests for binomial, and Wilcoxin rank sum tests for categorical variables were used. Regression models were used to investigate the training effect on score changes from baseline to post-tests. Baseline test scores and concussion history are controlled in the regression model. Ordinal logistic regression is conducted for test Highest 3 Second Hold (CCFT) test.

### Example 2: Results of Sensorimotor Activity Training to Improve Functional Performance of Soccer Athletes

There were 78 athletes enrolled from MC, and 52 from UWA. Across all variables reported, there were no differences between the athletes in the experimental and control at baseline. Approximately 44% of the participants at each school were female and a majority of the sample were white (84.6% at MC and 68.6% at UWA; p=0.056). At MC, 28.2% reported a history of previous concussion, 42.3% at UWA (p=0.096). Of the participants, 64.1% at MC and 61.5% at UWA acknowledged a history of prior lower extremity injury. The mean age of the participants was 20 years (p=0.796).

Regression results for the training activities and functional performance measures are presented in Table 4. The table presents the name of the activity or functional performance evaluation and whether it was measured in the virtual environment or as a traditional clinical test. The coefficient estimates provide the mean improvement in score between baseline and post-intervention testing for the individuals in the experimental group compared to individuals in the control group. The p-value indicates significance, values <0.05 denote a significant difference of the change score between the control and experimental pre- to post-intervention.

TABLE 4

Regression Results

| Tests | Coefficient Estimates (Std Err) | P-value |
|---|---|---|
| VR Scores | | |
| Sensorimotor Activities | | |
| Smooth Pursuits | 74.5 (14.6) | <0.0001 |
| Saccades | 94.30 (13.4) | <0.0001 |
| Near Point Convergence | 37.46 (14.4) | 0.01 |
| Peripheral Vision | 44.85 (8.4) | <0.0001 |
| Cervical Proprioception | 41.59 (10.9) | 0.0002 |
| Cervical Neuromotor Control | 43.5 (10.4) | <0.0001 |
| Visual Figure | 122.71 (12.9) | <0.0001 |
| Clinical Outcomes | | |
| Sensorimotor Activities | | |
| CCFT3 (mmHg) | 0.90 (0.2) | <0.0001 |
| DNFET (Seconds) | 8.61 (3.5) | 0.016 |
| Total Balance Score | 5.96 (1.5) | 0.0002 |
| Feet together | 6.92 (2.2) | 0.002 |
| Tandem - Right | 7.21 (2.5) | 0.005 |
| Tandem - Left | 4.46 (1.8) | 0.013 |
| Single-leg - Right | 8.57 (3.1) | 0.006 |
| Single-leg - Left | 9.70 (2.9) | 0.001 |
| Inspection Time | 1.86 (0.8) | 0.018 |

For the immersive virtual sensorimotor activities, there was a significant training effect for all sensorimotor activity proficiencies for the participants in the experimental group compared to the control (p values between 0<0.0001 and 0.01). For the functional performance evaluation, visual figure, there was a significant improvement in the experimental group (p<0.0001), and 122.71 point increase in performance of the experimental group over the performance pre-to post of the control. For the clinical outcome measures, there were significant improvements in cervical neuromotor control (CCFT3; p<0.0001) and endurance (DNFET; p=0.016).

Each of the balance stances produced a significant improvement, as well as the total balance score in the experimental compared to the control at post-testing (p-values between 0.0002 and 0.001). For inspection time, there was a significant improvement in reaction speed for the experimental group (p=0.018).

The results indicate significant effects in all immersive virtual sensorimotor training activity proficiencies and functional performance measures for the experimental group compared to the control. Second, there were also significant improvements in the clinical tests of cervical neuromotor control and endurance, standing balance, and inspection time (both in and out of the virtual environment). These findings indicate that there is a transfer effect of the sensorimotor training activities to clinically based measures of functional performance.

### Example 3: Study Design for Sensorimotor Activity Training of Sensorimotor Control Affecting Injury Incidence of Collegiate Soccer Players

Given the incidence of concussion and the number of exposures each athlete accumulates across years of play, many collegiate athletes have suffered one or more concussions that disrupted their sensorimotor control prior to beginning college. The applicant determined the effectiveness of a multi-modal sensorimotor intervention, using traditional training techniques and immersive virtual sensorimotor activities, on injury reduction in collegiate athletes.

Trial design: This study was conducted as a pilot, one-arm intervention using, 1) pre- and post-participation measures and 2) injury incidence rate over the 2018 sports-season compared to a historical control, identified as the 2017 season of the same teams. This research protocol was registered prospectively on clinicaltrials.gov and there were no changes to the methods after trial commencement. The study was approved by the University of Mississippi Institutional Review Board (IRB) and the Mississippi college IRB prior to initiation. Informed written consent was received prospectively and the rights of each participant was protected.

Participants: Participants were recruited from the men’s and women’s soccer teams at Mississippi College in Clinton, MS, USA, a Division II member of the NCAA, upon reporting to campus for the 2018 season on Aug. 11, 2018. Athletes eligible to play in the 2018 soccer season, aged 18 or over, were eligible for inclusion. Athletes with a current diagnosis of concussion (i.e. non-medically cleared), lower-extremity musculoskeletal injury, or other diagnosis that would prevent the athlete from participating in the intervention were excluded. In addition, an athlete sustaining a concussion during the timeframe for the intervention delivery would be withdrawn.

Multi-Modal Sensorimotor Training Intervention: The intervention was 4-weeks long, consisting of 2 face-to-face sessions per week. Within the intervention, participants completed an array of training activities, aiming at the various sub-systems which contribute to overall sensorimotor control. These training activities included vestibular, visual and oculomotor activities, cervical neuromotor control and strength training, and postural/balance exercises. The equipment utilized, a basic description of each function of sensorimotor control, and the specific abilities addressed during the training sessions are in Table 4. Part of the intervention included four novel activities/games in virtual reality (VR) to train several components of oculomotor control and visual acuity. These were delivered via VR HMD.

TABLE 5

Overview of training and equipment utilized for the intervention delivery

| | | |
|---|---|---|
| Functional Category | Specific Abilities Trained | Equipment Used |
| Oculomotor control | Smooth Pursuit | Headset Virtual Reality |
| | Saccades | Headset Virtual Reality Eye charts/ letter boards |
| | Convergence | Dice on a table Visual stimulus presented on a smart phone |
| Visual Acuity | Peripheral Perception | Headset Virtual Reality |
| | Dynamic Gaze Stability | Visual stimulus presented on a smart phone Eye charts |
| | Figure Ground Discrimination | Headset Virtual Reality |
| Cervical Neuromotor Control | Proprioceptive accuracy | Bulls-eye Targets (Tracker Laser) Head-mounted LED lights |
| | Kinesthetic Awareness | Maze and Target Posters (SenMoCor System) Head-mounted LED lights |
| | Deep Flexor Activation | Stabilizer Pressure Biofeedback (Chattanooga) Towel rolls Portable Mat tables Airex Yoga Mats |
| | Deep Extensor Activation | Towel rolls Portable Mat tables Airex Yoga Mats |
| | Functional Cervical Control/ Strength | IronNeck with appropriate resistance (5 lb. - 40 lb.) |
| Coordination | Head-Eye Coordination | Visual stimulus presented on a smart phone or on a wall |
| | Sport-Specific Coordination | Soccer ball Visual stimulus on each player |
| Postural Control | Standing Balance (completed during performance of the above activities) | BOSU Elite Airex foam pads |

The exercises delivered face to face were completed in a group format, using a standardized circuit of exercises that each athlete completed. A typical session included 2-3 rounds of 5-6 exercise stations for 2.5-3.5 minutes at each station. Each session lasted approximately 45 minutes. The sessions continued for 4 weeks, into the start of the official season. Subsequent sessions built upon previous sessions to work the sensorimotor control system in progressively more challenging and sport-specific scenarios.

Although the standardized exercises were delivered in a group format, the monitors at each station provided tailoring to ensure an appropriate challenge for each individual. Generally, this involved changing the stance position (e.g. double leg to single leg stance) and/or the standing surface (e.g. firm ground to foam or BOSU surface).

Clinical Measures: A description of the clinical measures of sensorimotor control used in this project can be found below. These outcomes were chosen because they have been extensively used in research related to concussion to measure various domains of sensorimotor control and have demonstrated clinical usefulness and psychomotor soundness.

Static Balance was measured as follows. The Sway Balance App is a Food and Drug Administration approved medical device with research literature supporting its use as a valid and reliable baseline assessment of static balance in athletes. Sway uses the triaxial accelerometer in a smartphone and a proprietary algorithm to calculate postural sway on a 0 - 100 point scale, where 100 is perfect. Participants completed this test by logging in to the Sway App on their phone. The total balance score, calculated across three separate trials, was recorded.

Near Point Convergence (NPC) was measured as follows. NPC was tested by asking each participant to follow the path of a bead threaded on a string and marked with an 11-point font “x”, with both eyes as it moved horizontally towards his/her nose. The participant was instructed to stop the bead along the string where either: 1) diplopia of the “x” was reported or, 2) the examiner observed one eye moving laterally away from midline. The test was completed 3 times and the mean distance of NPC was calculated and recorded in centimeters.

Cranial Cervical Flexion Test (CCFT3 and CCFT10) was measured as follows. Cranial Cervical Flexion Test was used as a test of cervical flexor control and endurance by having the participant recruit the deep neck flexors in a precise manner according to the Stabilizer biofeedback device (Chattanooga). As described by Jull,43 this test had 2 stages. Stage 1 (CCFT3): was used to measure the ability to recruit and control the deep cervical flexors. The highest level (22, 24, 26, 28 or 30 mmHg) that the participant could achieve and hold for 3 seconds with the correct muscle action, without palpable activity of the superficial flexors was recorded. Stage 2 (CCFT10): was used as a test of isometric endurance of the DCF. This stage was initiated with every participant that completed the correct movement of craniocervical flexion in stage 1, even if all pressures were not reached. At each step, the test was progressed to the next pressure target if the participant performed 3 repetitions of 10-second holds without substitution strategies. The highest level (22, 24, 26, 28 or 30 mmHg) that the participant could achieve and hold for three, 10-second counts with the correct muscle action was recorded.

Joint Position Error Test (JPE) was measured as follows. The JPE was utilized as a test of cervical proprioception and motor control. The subject’s ability to relocate the natural head posture following active cervical movements (left rotation, right rotation and extension) was tested without the use of vision. With the participant wearing a blindfold and a laser light attached to a headband, an examiner guided the participant to the center of the target using the light. Each direction was tested 3 times, in series with the examiner passively repositioning the participant’s head to the center of the target in between each repetition. Using a calibrated target with numbered lines (Tracker Target), performance was recorded to the nearest 0.5, with the examiner rounding up when in question. The mean absolute error across all 9 trials was calculated and reported in centimeters.

Injury Incidence Rate: The second primary outcome was injury incidence rate during the 2018 soccer season compared to a historical control (2017 season). For both years of interest, injury was defined using the same criteria, according to the NCAA-ISP, and included concussions and traumatic lower extremity musculoskeletal injuries. In this study, a reportable injury was one that (1) occurred during participation in a NCAA-sanctioned practice or competition and (2) required attention from an athletic trainer and/or physician. Contusions and non-traumatic (i.e. overuse) injuries were not included. The rate of injury was based on athlete exposures (AE). Each AE was defined as one student-athlete participating in one NCAA-sanctioned practice or competition which exposed the athlete to the possibility of injury, regardless of the time associated with that participation. Athlete exposures were counted as reported in the Countable Athletic Related Activities (CARA) logs utilized by NCAA sanctioned schools. The participating university collected athlete injuries and tracked CARA activities per their standard of practice. The systems used to collect and report these data were the same in 2017 and 2018.

Statistical Methods: Means with standard deviations and counts with percentages were used as descriptive statistics for baseline characteristics. Right skewed outcomes (PCSS, NPC, DVA, JPE) were modeled with log-gamma mixed effect random intercept models with variance component covariance structures. Normal outcomes (static balance) were modeled with Gaussian random intercept models. Finally, count outcomes (PCSS, CCFT) were modeled with Poisson generalized estimating equations or mixed models. Injury rates were modeled using Poisson models, as well. Two models for each outcome were constructed: one interacting pre/post intervention with previous concussion status and one pooling across and adjusting for previous concussion status. Models were translated to expected marginal outcomes and differences in expected marginal outcomes for ease of interpretability. All models were adjusted for sex, age, race, and lifetime number of years of soccer played. All analyses were conducted in Stata v15.1 (StataCorp, College Station, TX).

### Example 4: Sensorimotor Activity Training Leading to Reduced Injury Incidence of Collegiate Soccer Players

All of the eligible athletes (n=75; 38 males and 37 females), including 30 athletes with a history of concussion, enrolled in the research study. Of these, 72 received the intended treatment and were included in the analyses. Three participants dropped out of the study concurrent to their decision to stop playing soccer and no outcome data could be obtained. Baseline testing was completed on Aug. 12, 2018. The training intervention began on Aug. 13, 2018, concluded on Sep. 7, 2018, with post-intervention testing completed on September 10 and 12, 2018. Baseline characteristics can be found in Table 3. The mean age of the participants was 20.2 (SD=1.46). Thirty-five (49%) of the final cohort were females, and 16 (22%) were non-Caucasian. Twenty-eight (39%) had experienced a prior concussion, and the mean number of years of soccer played was 13.9 (SD=2.31).

Treatment delivery off the 72 participants who completed baseline and post-participation outcome assessments, 64 (88.9%) completed all 8 intervention sessions as scheduled; 8 (11.1%) completed seven intervention sessions as scheduled, missing one training session.

As presented in Table 6, according to the statistical analyses, the intervention was effective in improving static balance, NPC, and CCFT3 and CCFT10 values with similar improvement in both groups. As measured by the Sway Balance App, there was an increase of 3.8 points on average in static balance between baseline and post-testing (88.4 vs. 92.2; p<0.01). The distance of NPC decreased by 0.63 centimeters (1.69 vs. 1.06; p<0.01). There was also an improvement in motor control and endurance of the deep-cervical flexors, as measured by the CCFT3 and CCFT10 by averages of 2.6 and 4.3 mmHg, respectively (25.9 vs. 28.5; p<0.01 and 23.5 vs. 27.8; p<0.01).

TABLE 6

Regression Estimates

| Not Previously Concussed | | | | Previously Concussed | | |
|---|---|---|---|---|---|---|
| | Pre-Int | Post-Int | Difference | Pre-Int | Post-Int | Difference |
| Static Balance | 88.2 | 91.2 | 2.97 p=0.006 | 88.6 | 93.7 | 5.06 p<0.001 |
| | (86.2, 90.2) | (89.1, 93.2) | (0.83, 5.11) | (86.1, 91.2) | (91.1, 96.3) | (2.39, 7.72) |
| NPC | 1.78 | 1.20 | -0.57 p=0.031 | 1.56 | 0.86 | -0.70 p=0.019 |
| | (1.16, 2.39) | (0.71, 1.70) | (-1.10, -0.05) | (0.88, 2.24) | (0.36, 1.36) | (-1.28, -0.11) |
| CFFT 3 | 25.6 | 28.4 | 2.82 p<0.001 | 26.3 | 28.6 | 2.31 p=0.022 |
| | (24.6, 26.6) | (27.2, 29.6) | (1.29, 4.34) | (25.0, 27.6) | (27.1, 30.2) | (0.33, 4.28) |
| CFFT 10 | 23.2 | 27.7 | 4.54 p<0.001 | 23.9 | 27.9 | 3.96 p<0.001 |
| | (22.5, 23.9) | (26.6, 28.9) | (3.23, 5.85) | (22.9, 25.0) | (26.4, 29.4) | (2.26, 5.67) |
| JPE | 4.09 | 4.07 | -0.02 p=0.933 | 4.54 | 3.54 | -1.00 p<0.001 |
| | (3.72, 4.47) | (3.70, 4.45) | (-0.46, 0.42) | (4.02, 5.06) | (3.13, 3.95) | (-1.55, -0.45) |

For absolute error on the JPE, there was an interaction effect, with the training significantly benefitting the previously concussed group (4.54 vs. 3.54; p<0.01) and no change in the non-previously concussed group (p=0.93). Finally, we did observe a 27% total reduction in injury rates after treatment (11.8 per 1000 athlete exposures in 2017 vs. 8.94 per 1000 athlete exposures in 2018), but this did not reach statistical significance (p=0.18).

The marginal expected values for Cranial Cervical Flexion Test 10 second hold (CCFT10) were modeled using generalized estimating equations with Poisson families, adjusted for sex, age, race, and number of years of soccer played. The p-value for interaction between pre/post and concussion status was p=0.373, and pooling across concussion status yielded expected marginal CCFT10s of 23.5 and 27.8, pre and post, respectively (p<0.001).

The marginal expected values for static balance as measured by the Sway balance app were modeled using multilevel mixed models with Gaussian families, adjusted for sex, age, race, and number of years of soccer played. The p-value for interaction between pre/post and concussion status was p=0.232, and pooling across concussion status yielded expected marginal Sway score of 88.4 and 92.2, pre and post, respectively (p<0.001).

The results indicate that an intervention, such as the one described here, improved clinical measures of sensorimotor control, potentially decreasing the risk of sports-injury for individuals with and without a history of concussion. This study is the first description of training activities developed to target each sub-system component of sensorimotor control as a comprehensive population-based intervention to target primary and tertiary prevention in athletes. Additionally, this is the first description of the use of a virtual immersive environment for the purposes of sensorimotor training.

The field of sports-performance training uses various methods to improve upon reaction time, balance, speed and other abilities in healthy athletes. As an extension of sports-performance and as a form of primary injury prevention for those with no history of concussion, the intervention was presumed to fine-tune a stable system. On the other hand, because deficits in sensorimotor and neuromotor control are potential contributors to the increase risk of secondary injury after an initial concussion, the intervention was used to rehabilitate latent and subtle deficits (tertiary injury prevention).

In addition to the significant improvements demonstrated in the clinical measures of sensorimotor control shown by increased proficiencies, the results demonstrate a reduction in rate of injury between the 2018 and 2017 season and a 27% reduction in total injury incidence. This downward trend promotes optimism in the ability of the intervention to control an underlying neural mechanism contributing to injury risk, thereby decreasing incidence.

This research showed the use of sensorimotor activity training for injury reduction in athletes. It is important to emphasize that the participants were healthy athletes at the time the study began. This point is important because the outcomes utilized are typically used to identify impairment of the visual, vestibular, neuromotor, and postural control systems. Most of the athletes performed within normal ranges or at the ceiling at baseline, leaving little room for improvement. The results demonstrate improvement in static balance, NPC, and cervical neuromotor control and endurance (CCFT 3 and CCFT10).

### Conclusion

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

We claim:

1. A method for improving a selected functional performance of a user in the real world through sensorimotor training provided in a virtual immersive environment, the method comprising:
(a) measuring the selected functional performance of the user and recording a baseline functional performance metric of the user;
(b) providing a computer system configured to display the virtual immersive environment to the user, the computer system including
a head-mounted display device for immersing the user in the virtual immersive environment,
one or more position/motion sensors for recording movement data of the user within the virtual immersive environment,
one or more processors, and
one or more hardware storage devices;
displaying a virtual immersive environment to the user by way of the head-mounted display;
(c) placing the head-mounted display device over the user’s head so that the display is visible to the user;
(d) providing instructions directing the user to physically execute two or more sensorimotor activities within the virtual immersive environment selected to improve the selected functional performance of the user, wherein the two or more sensorimotor activities comprise two or more of smooth pursuit, saccade, near point convergence, peripheral vision acuity, eyes-then-head, gaze stability, cervical proprioception and kinesthetic awareness, or cervical neuromotor control, at least one of the sensorimotor activities comprising the user tracking an object that moves within the virtual immersive environment in an unpredictable movement pattern and remains visible while moving in the unpredictable movement pattern; and
(e) recording movement data of the user during execution of the two or more sensorimotor activities including measuring at least one of eye position or movement of one or both eyes in response to sensory stimuli;
(f) based on the recorded movement data, determining a baseline sensorimotor activity metric of the user indicating the user’s baseline proficiency in executing the two or more sensorimotor activities;
(g) providing additional instructions directing the user to repeat execution of the two or more sensorimotor activities within the virtual immersive environment, and recording further movement data of the user during the repeated execution of the two or more sensorimotor activities;
(h) based on the recorded further movement data, determining a trained sensorimotor activity metric of the user indicating the user’s proficiency in executing the two or more sensorimotor activities following repeated execution of the two or more sensorimotor activities;
(i) comparing the trained sensorimotor activity metric to the baseline sensorimotor activity metric; and
(j) repeating steps (d) and (e) to provide until an increase in proficiency in performing the two or more sensorimotor activities within the virtual immersive environment is determined;
(k) upon determining an increase in proficiency in performing the two or more sensorimotor activities within the virtual immersive environment, measuring the selected functional performance of the user again, in the real world, and recording a trained functional performance metric of the user,
(l) wherein the method improves the selected functional performance of the user in the real world as a result of increased proficiency by the user in performing the two or more sensorimotor activities within the virtual immersive environment,
(m) wherein the improved functional performance of the user in the real world includes improved proficiency by the user in at least one activity that is outside of the virtual immersive environment, and
(n) wherein the improved functional performance of the user in the real world is different than the increased proficiency by the user in performing the two or more sensorimotor activities within the virtual immersive environment.

2. The method of claim 1, wherein the instructions, the additional instructions, or both are provided to the user as audio or visual instructions.

3. The method of claim 1, wherein the step of recording movement data, the step of recording the further movement data, or both comprise monitoring two or more of head movement data, hand movement data, or eye movement data.

4. The method of claim 1, wherein the virtual immersive environment covers substantially all of the user’s visual field.

5. The method of claim 1, wherein the one or more position/motion sensors comprise a hand tracking sensor for tracking position and movement of the user’s hand within the virtual immersive environment relative to other objects displayed within the virtual immersive environment.

6. The method of claim 1, wherein the one or more position/motion sensors comprise an accelerometer head sensor, a gyroscope head sensor, an eye tracking device, a magnetometer, or combination thereof.

7. The method of claim 1, wherein the method improves functional performance of the user outside of the virtual environment by improving one or more of a multi-joint or whole-body motor response, cervical neuromotor control, balance, coordination, or agility.

8. The method of claim 7, wherein functional performance is measured by evaluating one or more of: engagement of multiple senses; cognitive processes; or complex motor responses of the user.

9. The method of claim 1, wherein the additional instructions directing the user to repeat execution of the one or more sensorimotor activities within the virtual immersive environment progress in difficulty over time through adjustment of one or more difficulty parameters within the virtual immersive environment.

10. The method of claim 9, wherein the progress in difficulty through adjustment of one or more difficulty parameters within the virtual immersive environment includes a modification of target motion that increase in the required level of head movement.

11. The method of claim 9, wherein the one or more sensorimotor activities within the virtual immersive environment progress in difficulty over time by adjustment to target object speed, target object size, target object number, target object path, level of environmental distractions, or combination thereof.

12. The method of claim 1, wherein the additional instructions directing the user to repeat execution of the one or more sensorimotor activities direct the user to stand on an unstable surface, in a variable standing position, or both.

13. The method of claim 1, wherein the user is neurologically impaired due to trauma, aging, or both.

14. A method for improving a selected functional performance of a user through sensorimotor training provided in a virtual immersive environment, the method comprising:
(a) measuring the selected functional performance of the user and recording a baseline functional performance metric of the user, wherein the selected functional performance includes one or more of a multi joint or whole-body motor response, cervical neuromotor control, balance, coordination, or agility;
(b) providing a computer system configured to display the virtual immersive environment to the user, the computer system including
a head-mounted display device for immersing the user in the virtual immersive environment,
one or more position/motion sensors for recording movement data of the user within the virtual immersive environment,
one or more processors, and
one or more hardware storage devices,
(c) placing the head-mounted display device over the user’s head so that the display is visible to the user;
(d) displaying a virtual immersive environment to the user by way of the head-mounted display, the virtual immersive environment comprising a first object configured to move within the virtual immersive environment based on head movement of the user, and comprising a second object that remains visible within the virtual immersive environment and has an unpredictable movement pattern within the virtual immersive environment;
(e) providing instructions directing the user to physically execute two or more sensorimotor activities within the virtual immersive environment selected to improve the selected functional performance of the user, at least one of the sensorimotor activities comprising the user moving an object within the virtual immersive environment by way of head movement, and at least one of the sensorimotor activities comprising the user tracking an object that moves in an unpredictable movement pattern and remains visible while moving in the unpredictable movement pattern; and
(f) recording movement data of the user during execution of the two or more sensorimotor activities;
(g) based on the recorded movement data, determining a baseline sensorimotor activity metric of the user indicating the user’s baseline proficiency in executing the two or more sensorimotor activities;
(h) providing additional instructions directing the user to repeat execution of the two or more sensorimotor activities within the virtual immersive environment, and recording further movement data of the user during the repeated execution of the two or more sensorimotor activities;
(h) based on the recorded further movement data, determining a trained sensorimotor activity metric of the user indicating the user’s proficiency in executing the two or more sensorimotor activities following repeated execution of the two or more sensorimotor activities;
(j) comparing the trained sensorimotor activity metric to the baseline sensorimotor activity metric; and
(k) repeating steps (e) and (f) to improve proficiency in performing the functional performance of step (a);
upon determining an increase in proficiency in performing the two or more sensorimotor activities within the virtual immersive environment, recording a trained functional performance metric of a user, wherein the method improves the selected functional performance of the user, including improving one or more of a multi joint or whole-body motor response, cervical neuromotor control, balance, coordination, or agility as a result of increasing in proficiency in performing the two or more sensorimotor activities within the virtual immersive environment.

15. The method of claim 14, wherein the two or more sensorimotor activities comprise two or more of smooth pursuit, saccade, near point convergence, peripheral vision acuity, eyes-then-head, gaze stability, cervical proprioception and kinesthetic awareness, or cervical neuromotor control.

16. A method for treating or preventing concussion injury by improving a selected functional performance of a user through sensorimotor training provided in a virtual immersive environment, the method comprising:
measuring the selected functional performance of the user outside of the virtual immersive environment and recording a baseline functional performance metric of the user;
providing a computer system configured to display the virtual immersive environment to the user, the computer system including
a head-mounted display device for immersing the user in the virtual immersive environment,
one or more position/motion sensors for recording movement data of the user within the virtual immersive environment,
one or more processors, and
one or more hardware storage devices;
placing the head-mounted display device over the user’s head so that the display is visible to the user
displaying a virtual immersive environment to the user by way of the head-mounted display, the virtual immersive environment including an object configured to move in any three-dimensional direction within the virtual immersive environment;
providing instructions directing the user to execute two or more sensorimotor activities within the virtual immersive environment selected to improve the selected functional performance of the user outside of the virtual immersive environment, wherein the two or more sensorimotor activities comprise two or more of smooth pursuit, saccade, near point convergence, peripheral vision acuity, eyes-then-head, gaze stability, cervical proprioception and kinesthetic awareness, or cervical neuromotor control, at least one of the sensorimotor activities comprising the user tracking the object;
recording movement data of the user during execution of the two or more sensorimotor activities;
based on the recorded movement data, determining a baseline sensorimotor activity metric of the user indicating the user’s baseline proficiency in executing the two or more sensorimotor activities;
providing additional instructions directing the user to repeat execution of the two or more sensorimotor activities within the virtual immersive environment, and recording further movement data of the user during the repeated execution of the two or more sensorimotor activities;
based on the recorded further movement data, determining a trained sensorimotor activity metric of the user indicating the user’s proficiency in executing the two or more sensorimotor activities following repeated execution of the two or more sensorimotor activities;
comparing the trained sensorimotor activity metric to the baseline sensorimotor activity metric; and
upon determining an increase in proficiency in performing the two or more sensorimotor activities within the virtual immersive environment, recording a trained functional performance metric of a user outside the virtual immersive environment,
wherein the method treats or prevents concussion injury by improving the selected functional performance of the user outside the virtual immersive environment as a result of increased proficiency by the user in performing the two or more sensorimotor activities within the virtual immersive environment, and
wherein the functional performance improved by the method is different than the two or more sensorimotor activities performed within the virtual immersive environment.

17. The method of claim 16, wherein the step of recording movement data, the step of recording the further movement data, or both comprise monitoring two or more of head movement data, hand movement data, or eye movement data.

18. The method of claim 1, wherein the virtual immersive environment is three dimensional, and wherein the object is also configured to move toward and away from the user within the virtual immersive environment.

19. The method of claim 1, wherein the virtual immersive environment is three dimensional, and wherein the smooth pursuit sensorimotor activity comprises the object moving in all directions and with varying speeds while the user is directed to follow the path of the object by tracing the path of the target.

20. The method of claim 1, wherein the virtual immersive environment is three dimensional, and wherein the saccade sensorimotor activity comprises the object appearing within the virtual immersive environment while the user is directed to move their eyes toward the object, wherein upon selection of the object by the user, the object disappears and reappears in a different location within the virtual immersive environment.
